(19) United States
(12) Patent Application Publication
(10) Pub. No.: US 2022/0008440 A1
(43) Pub. Date: Jan. 13, 2022

(54) TEAT DISINFECTANT COMPOSITION

(71) Applicant: DeLaval Holding AB, Tumba (SE)

(72) Inventors: Lauren Hastings, Kansas City, MO (US); Sarah Leibowitz, Kansas City, MO (US); James Partridge, Kansas City, MO (US)

(21) Appl. No.: 17/297,100

(22) PCT Filed: Dec. 5, 2019
(86) PCT No.: PCT/SE2019/051232

§ 371 Date: May 26, 2021

(30) Foreign Application Priority Data
Dec. 5, 2018 (SE) 1851515-5

(51) Int. Cl. A61K 31/60; A61K 31/19; A61K 33/08; A61K 9/00; A61P 31/02

(52) U.S. Cl. CPC A61K 31/60; A61K 31/19; A61P 31/02; A61K 9/0017; A61K 33/08

(57) ABSTRACT

Antimicrobially effective compositions comprising lactic acid, salicylic acid, and at least one anionic surfactant are provided. The compositions are effective in controlling pathogen levels, particularly those commonly found on bovine teats to prevent or reduce the incidence of mastitis in lactating cows. In particular, the compositions possess germicidal and/or yeasticidal characteristics. Methods of using the antimicrobial compositions, as a part of a good milking routine, are also provided in which the compositions are applied to bovine teats.

### BACKGROUND OF THE INVENTION

### Field of the Invention

The present invention is generally directed toward a teat disinfectant composition that possesses yeasticidal functionality in addition to the antibacterial characteristics that are expected with teat disinfectants. Embodiments of the present invention comprise at least two organic acids and an anionic surfactant.

### Description of the Prior Art

Antimicrobial compositions are regularly employed to control or prevent pathogenic diseases in animals, especially mastitis in lactating bovines. Mastitis generally results from contact of the bovine mammary gland with pathogenic microorganisms. While it is most common for mastitis to be a result of a bacterial infection, mastitis also can be caused by yeasts or fungi. Regulatory changes in Europe now require that teat disinfectants possess yeasticidal functionality in addition to bactericidal functionality.

Traditional oxidative germicides like iodine and chlorine dioxide are yeasticidal, but are not ideal for use in some situations. For example, these active ingredients may not be desired in some markets because of residue concerns for iodine or chlorate.

Some current commercial teat dips do not kill yeast. For example, U.S. Pat. No. 9,750,755 describes a germicidal composition that comprises lactic acid and the anionic surfactant sodium octane sulfonate. However, this composition does not possess the yeasticidal functionality required by current European regulations. In addition, various components which comprise some germicidal compositions are manufactured in facilities that produce a wide range of compounds. Certain of these additional compounds may contaminate the components used in the germicidal compositions, which has been deemed unacceptable for a variety of reasons. Therefore, certain traditional germicidal composition components may need to be avoided depending upon the facility in which they are produced.

Accordingly, there is a need in the art to provide a germicidal composition that is effective in controlling, inhibiting, or otherwise reducing the incidence of mastitis in lactating bovines caused by bacteria and/or yeast.

### SUMMARY OF THE INVENTION

According to one embodiment of the present invention there is provided a germicidal and/or yeasticidal composition comprising lactic acid, salicylic acid and at least one anionic surfactant. Preferably, the anionic surfactant is selected from the group consisting of polyoxyethylene alkyl ether phosphates, sodium lauryl sulfate, and sodium lauryl ether sulfate.

In preferred embodiments, the antimicrobial composition comprises from 0.75% to 10% by weight of lactic acid, from 0.01% to 5% by weight of salicylic acid, and from 0.1% to 15% by weight of at least one anionic surfactant selected from the group consisting of polyoxyethylene alkyl ether phosphates, sodium lauryl sulfate, and sodium lauryl ether sulfate.

In certain embodiments, antimicrobial compositions according to the present invention produce at least a 5-log reduction in bacteria levels when tested according to EN 1656 and at least a 4-log reduction in yeast levels when tested according to EN 1657.

According to another embodiment of the present invention there is provided a teat dip composition for use in preventing mastitis in bovines that is caused by bacteria and/or yeast. The teat dip comprises lactic acid, salicylic acid and at least one anionic surfactant. The teat dip composition is applied to the teats of the bovines and is effective in reducing the levels of bacteria and/or yeast present on the bovine's teats. In preferred embodiments, the teat dip composition comprises from 0.75% to 10% by weight of lactic acid, from 0.01% to 5% by weight of salicylic acid, and from 0.1% to 15% by weight of at least one anionic surfactant selected from the group consisting of polyoxyethylene alkyl ether phosphates, sodium lauryl sulfate, and sodium lauryl ether sulfate.

According to still another embodiment of the present invention there are provided methods for preventing or otherwise reducing the incidence of mastitis in bovines that is caused by bacteria and/or yeast. The methods comprise applying to the teats of the bovine, preferably post milking, a germicidal and/or yeasticidal composition comprising lactic acid, salicylic acid and at least one anionic surfactant. The composition may be applied by any method that is conventional in the art, such as spraying, brushing, dipping or foaming of the animal's teats. Preferably, the composition comprises from 0.75% to 10% by weight of lactic acid, from 0.01% to 5% by weight of salicylic acid, and from 0.1% to 15% by weight of at least one anionic surfactant selected from the group consisting of polyoxyethylene alkyl ether phosphates, sodium lauryl sulfate, and sodium lauryl ether sulfate. The compositions preferably produce at least a 5-log reduction in bacteria levels when tested according to EN 1656 and at least a 4-log reduction in yeast levels when tested according to EN 1657.

### DETAILED DESCRIPTION OF THE PREFERRED EMBODIMENT

Compositions according to embodiments of the present invention possess germicidal and/or yeasticidal characteristics and are suitable for application to animal skin, especially bovine teats to prevent and/or reduce the incidence of mastitis in cattle.

As used herein, the term “germicidal” refers to the ability of a composition to destroy microorganisms, especially pathogenic organisms such as bacteria, and may or may not include bacterial spores.

As used herein, the term “yeasticidal” refers to the ability of a composition to destroy yeast, a type of fungus. Hence, if a composition possesses yeasticidal characteristics, it may also be referred to as fungicidal. A yeasticidal (fungicidal) composition may or may not destroy both vegetative and spore forms of yeast (fungi).

Certain embodiments according to the present invention comprise, consist of, or consist essentially of at least two germicidal agents, and at least one anionic surfactant. Additionally, certain embodiments may further comprise, consist of, or consist essentially of at least one of the following: magnesium oxide, one or more non-ionic surfactants, a thickener or film-forming agent, a buffer system, a dye or colorant, and one or more skin conditioning agents. These compositions, which are preferably in the form of a solution, are effective in controlling bacteria and yeast when used on lactating cows as a part of a good milking routine.

Embodiments of the present invention generally comprise two germicidal agents, both of which may be organic acids. In particularly preferred embodiments, one of the organic acids is salicylic acid and at least one other of the organic acids is lactic acid. The salicylic acid may be present in embodiments of the present invention in an amount of from 0.01% to 5% by weight, from 0.05% to 2.5% by weight, or from 0.1% to 1% by weight. The lactic acid may be present in embodiments of the present invention in an amount of from 0.75% to 10% by weight, from 1% to 7.5% by weight, or from 2% to 5% by weight.

In some embodiments of the present invention, the use of certain organic acids, such a glycolic acid and formic acid is avoided. In those embodiments, the compositions are essentially free of glycolic and/or formic acid (i.e., comprises less than 0.1%, or less than 0.01%, by weight of the acid). Also, in certain embodiments, the compositions are essentially free of antimicrobially effective solvents, particularly benzyl alcohol and/or phenoxyethanol (i.e., comprises less than 0.1%, or less than 0.01%, by weight of the solvents).

Compositions according to the present invention preferably comprise at least one anionic surfactant that boosts the germicidal efficacy of the lactic and salicylic acids. Preferably, the at least one anionic surfactant is selected from the group consisting of polyoxyethylene alkyl ether phosphates, sodium lauryl sulfate (SLS), and sodium lauryl ether sulfate (SLES), with SLS being most preferred. In certain embodiments, this particular anionic surfactant is present in the antimicrobial composition in an amount of from 0.1% to 15% by weight, from 0.2% to 10% by weight, or from 0.5% to 7% by weight. In particular, when the anionic surfactant comprises a polyoxyethylene alkyl ether phosphate (e.g., MULTITROPE 1214 by Croda), the polyoxyethylene alkyl ether phosphate is present in the antimicrobial composition in an amount of from 0.1% to 10% by weight, from 0.5% to 5% by weight, or from 1% by weight to 4% by weight. When the anionic surfactant comprises SLS, the SLS can be present in the antimicrobial composition in an amount of from 0.1% to 5% by weight, from 0.2% to 3% by weight, or from 0.5% to 2% by weight. When the anionic surfactant comprises SLES, the SLES can be present in the antimicrobial composition in an amount of from 0.5% to 15% by weight, from 0.75% to 10% by weight, or from 1% to 5% by weight. It is noted that preferred embodiments of the preset invention are essentially free of certain anionic surfactants, such as sodium octane sulfonate (SOS) (i.e., comprise less than 0.1% by weight, or less than 0.01% by weight of the surfactant).

In certain embodiments, the compositions may further comprise one or more non-ionic surfactants. Preferably, the non-ionic surfactant is polyethoxylated polyoxypropylene block copolymer (poloxamer) including the PLURONIC brand of poloxamers commercialized by BASF, or a polyoxyethylene sorbitan monooleate, such as TWEEN 80 (polysorbate 80). The one or more non-ionic surfactants are generally present in an amount of from 0.01% to 5% by weight, from 0.05% to 2.5% by weight, or from 0.1% to 1% by weight. In certain embodiments, the non-ionic surfactant acts as a chelator in these antimicrobial solutions, prolonging the physical and chemical stability of the solutions. In addition, the presence of the non-ionic surfactant, especially the poloxamer, provides advantageous effects from a manufacturing standpoint in that the nonionic surfactant contributes to rapid solubilization of salicylic acid and/or magnesium oxide, which is described in greater detail below. The one or more non-ionic surfactants may also enhance the wetting and/or barrier characteristics of the compositions.

In certain embodiments, one or more additional anionic surfactants may also be present in the antimicrobial composition to act as wetting agents. Generally, though, these additional anionic surfactants do not appreciably affect the antimicrobial performance of the compositions. Exemplary additional anionic surfactants include sodium C14-C15 olefin sulfonate, such as BIO-TERGE AS-40 from Stepan, or sodium dioctylsulfosuccinate, such as AEROSOL OT from Cytec.

Certain embodiments of the present invention may also comprise additional barrier- or film-forming agents, which help the composition remain on the animal's skin in between milking cycles. Barrier- and film-forming agents may coat the animal's skin and physically cover the open end of the teat canal to prevent infiltration of pathogenic microorganisms. Exemplary barrier-forming agents that may be used with the present invention include pullulan and xanthan gum (which may also function as a thickener). These barrier-forming agents may be present in the antimicrobial compositions in an amount of from 0.01% to 5% by weight, from 0.05% to 2.5% by weight, or from 0.1% to 1% by weight.

Compositions according to the present invention may also include one or more skin-conditioning agents. Exemplary skin conditioning agents include sorbitol, lanolin, allantoin and propylene glycol. As explained below, propylene glycol, in conjunction with magnesium oxide, assists in providing low temperature stability for the antimicrobial compositions. The skin conditioning agents may be present in the compositions in an amount of from 1% to 30% by weight, from 5% to 25% by weight, or from 10% to 20% by weight. In certain embodiments, sorbitol is the majority component of the various skin-conditioning agents and can comprise from 5% to 20%, from 7.5% to 15%, or from 10% to 12.5% by weight of the composition. Lanolin may comprise from 0.5% to 8% by weight, from 1% to 6.5% by weight, or from 3% to 5% by weight of the composition. Propylene glycol may comprise from 0.5% to 10% by weight, from 1% to 7.5% by weight, or from 2.5% to 5% by weight of the composition.

The compositions according to the present invention may also include a dye so that extent of application of the compositions, especially to animal skin, is readily visually apparent. Exemplary dyes include E102 Granular and E133 Granular.

Certain compositions according to the present invention may further comprise one or more buffering agents. Preferred buffering agents can be either acids or bases that can be used to adjust the pH of the composition. Sodium hydroxide is a particularly preferred buffering agent. The buffering agent may be present in the composition in an amount of from 0.1 to 2.5% by weight, and preferably in an amount of from 0.2% to 1.5% by weight. Preferably, the antimicrobial compositions have a pH of from 2.5 to 3.5, and more preferably 2.8.

Aside from combinations of the aforementioned components, the balance of the composition comprises water, preferably distilled or deionized water. In certain embodiments, the compositions comprise at least 60%, at least 65%, or at least 70% by weight water, and no more than 95%, 90%, or 85% by weight water.

It is noted that a problem with the use of certain surfactants, such as SLS, and with the use of certain germicidal organic acids, such as salicylic acid, is that both may tend to fall out of solution at cold temperatures. Therefore, certain embodiments of the present invention may comprise additional components that function to keep these materials in solution. Magnesium oxide (MgO) and propylene glycol are included in the formula to provide low temperature stability, especially to keep SLS and salicylic acid in solution. Magnesium oxide is present within certain embodiments of the present invention in an amount of from 0.01% to 1% by weight, 0.025% to 0.5% by weight, or 0.05% to 0.25% by weight.

With respect to stability, certain compositions according to the present invention are highly storage stable across a wide range of storage conditions. By “storage stable” it is meant that the composition remains as a solution and that individual components do not separate or precipitate out of solution during the storage period. Certain compositions exhibit storage stability for at least 3 months, at least 6 months, or at least one year when stored at temperatures ranging from 4° C. to 40° C. Preferably, any test of the storage stability of the compositions is performed at a constant temperature over the storage period, the temperature being 4° C., 25° C., or 40° C.

In certain embodiments of the present invention, selection of the ratio of the anionic surfactant, and especially SLS, to the skin conditioning agent, especially propylene glycol, is important to achieving both low temperature stability and antimicrobial efficacy of the formulation. In preferred embodiments, the ratio of the anionic surfactant (e.g., SLS) to the skin conditioning agent (e.g., propylene glycol) is greater than 1:1, from 1:1.5 to 1:10, or from 1:2 to 1:5. In some embodiments, especially when SLS is present, the concentration of SLS should, at the same time, be no greater than 2%.

Preferred compositions according to the present invention exhibit germicidal and/or yeasticidal efficacy, and preferably both germicidal and yeasticidal efficacy. Measures of germicidal and yeasticidal efficacy include testing of the composition according to EN 1656 and EN 1657, respectively. These test procedures are described in further detail below. Preferably, when tested according to EN 1656, certain compositions according to the present invention produce at least a 5-log reduction in levels of one or more bacteria, including, but not limited to E. coli, S. aureus, and S. uberis. When tested according to EN 1657, certain compositions according to the present invention produce at least a 4-log reduction in levels of one or more yeasts, such as C. albicans. Preferably, the EN 1656 and EN 1657 testing is performed in the presence of an interfering substance, such as 1% reconstituted skim milk, 3% bovine albumin (a simulated “low soil” condition), or 10% bovine albumin and 10% yeast extract (a simulated “high soil” condition).

As mentioned previously, compositions according to the present invention can be applied to animal skin, especially bovine teats, in a therapeutic and/or prophylactic manner to prevent or reduce the incidences of bovine mastitis that is caused by bacteria and/or yeast. Thus, the compositions described herein can be used as a part of a well-established practice to improve animal hygiene and kill bacteria and/or yeast residing on the bovine's teats, which otherwise may infiltrate the teat orifice and potentially cause mastitis. Preferably, compositions according to the present invention are formulated as read-to-use compositions, which do not require further dilution prior to application to the animal's teats. In addition, the compositions are most suitable for use on bovine teats as a post-milking topical application. Application of the compositions may occur by any means conventional in the art including spraying, dipping, or foaming of the animal's teats.

The following Table 1 summarizes preferred compositions according to the present invention. It is understood that the more preferred and most preferred ranges for each component expressed in Table 1 are fully encompassed by the preferred ranges that component, and that any lower limit may be combined with any upper limit of any expressed range.

TABLE 1

| | | More | Most |
|---|---|---|---|
| | Preferred | preferred | preferred |
| | amount | amount | amount |
| Component | (wt. %) | (wt. %) | (wt. %) |
| Lactic acid | 0.75-10% | 1-7.5% | 2-5% |
| Salicylic acid | 0.01-5% | 0.05-2.5% | 0.1-1% |
| Anionic surfactant(s) | 0.1-15% | 0.2-10% | 0.5-7% |
| (total) | | | |
| SLS | 0.1-5% | 0.2-3% | 0.5-2% |
| (when present) | | | |
| SLES | 0.5-15% | 0.75-10% | 1-5% |
| (when present) | | | |
| Polyoxyethylene | 0.1-10% | 0.5-5% | 1-4% |
| alkyl ether phosphate | | | |
| (when present) | | | |
| MgO | 0.01-1% | 0.025-0.5% | 0.05-0.25% |
| Nonionic surfactant(s) | 0.01-5% | 0.05-2.5% | 0.1-1% |
| Skin conditioning agent(s) | 1-30% | 5-25% | 10-20% |
| (total) | | | |
| Propylene glycol | 0.5-10% | 1-7.5% | 2.5-5% |
| (when present) | | | |
| Ratio of SLS to propylene | <1:1 | 1:1.5 to 1:10 | 1:2 to 1:5 |
| glycol (when both present) | | | |

### EXAMPLES

The following tables describe compositions made in accordance with the present invention. These examples are provided by way of illustration and should not be taken as limiting upon the scope of the present invention. Certain formulations were tested according to several procedures to evaluate characteristics such as germicidal and yeasticidal efficacy.

### Microbiology Testing

Microbiology tests were performed to determine if some representative compositions would be capable of the standard log reduction requirement to be a bactericidal and yeasticidal agent. These tests involved exposing the bacteria or yeast to an interfering substance then exposing this mixture to the compositions. The log reduction obtained from these microbiology tests are provided in the tables, below.

EN1656—Chemical Disinfectants and Antiseptics—Quantitative suspension test for the evaluation of bactericidal activity of chemical disinfectants and antiseptics used in the veterinary field.

In this test, bacteria are exposed to an interfering substance before being exposed to the composition. The interfering substance used was milk (1% reconstituted skim milk). The microorganisms used to evaluate germicidal efficacy were Escherichia coli ATCC 10536 (E. coli), Streptococcus uberis ATCC 19436 (S. uberis), Staphylococcus aureus ATCC 6538 (S. aureus).

The microorganisms are prepared from glycerol stocks that are spread onto tryptic soy plates and allowed in incubate for 18-24 hours. This is the stock culture. A subculture is then prepared from the stock culture by streaking tryptic soy agar plates and allowing incubation for 18-24 hours. Second and third subcultures are prepared from the first subculture in the same way. These subcultures are the working cultures. Loopfuls of the working cultures are then transferred to diluent to create a standardized bacterial cell suspension, this suspension is used in the testing against the compositions. The bacteria were diluted to form a suspension having an initial concentration of about 10⁸ cfu/ml.

Skimmed milk (10 g/l) was used as the interfering substance. 1 ml of interfering substance and 1 ml of bacterial suspension were mixed and left in contact for 2 minutes at 30° C. 8 ml of the formulations described below in Tables 2-5 were then added to the mixture and left in contact for 5 minute at 30° C. For the inoculum control, one milliliter of the bacteria solution was removed and diluted with 9 ml of diluent at pH 7.0, and then four successive dilutions were made. Samples from each dilution were plated in duplicate and agar was added. For each treatment, one ml of the previous mixture was added to 9 ml of neutralizing solution and then mixed. One ml of the neutralized solution was then placed into petri dishes in duplicate. Approximately 15 ml of sterile tryptic soy agar was added to each Petri dish and when solidified, each plate was incubated at 36° C. for 48 hours. Colony forming units on plates were counted after 24 and 48 hours incubation. This procedure was repeated for all samples to be tested. Passing bactericidal efficacy is at least a 5-log reduction of starting inoculum cell counts.

EN1657—Chemical Disinfectants and Antiseptics—Quantitative suspension test for the evaluation of fungicidal or yeasticidal activity of chemical disinfectants and antiseptics used in the veterinary field.

In this test, yeasts are exposed to an interfering substance before being exposed to the composition. The interfering substance was milk (1% reconstituted skim milk). The microorganism used to evaluate teat disinfection was Candida albicans ATCC 10231 (C. albicans).

The microorganisms are prepared from glycerol stocks that are spread onto malt extract plates and allowed in incubate for 42-48 hours. This is the stock culture. A subculture is then prepared from the stock culture by streaking malt extract agar plates and allowing incubation for 42-72 hours. Second and third subcultures are prepared from the first subculture in the same way. These subcultures are the working cultures. Loopfuls of the working cultures are then transferred to diluent to create a standardized cell suspension, this suspension is used in the testing against the compositions. The yeasts were diluted to form a suspension having an initial concentration of about 10⁷ cfu/ml.

Standard general disinfection temperature is 10° C.; however, 30° C. is a more realistic teat disinfection temperature. All reagents used in the testing of the compositions are equilibrated to temperature before the testing begins. The standard contact time for general disinfection is 30 minutes; however, the practical contact times for teat disinfection is 5 minutes. To test the compositions from Tables 2-5, 1 ml of interfering substance and 1 ml of yeast extract were mixed and left in contact for 2 minutes at 30° C. 8 ml of the formulations described below in Tables 2-5 were then added to the mixture and left in contact for 5 minutes at 30° C. For the inoculum control, one milliliter of the yeast solution was removed and diluted with 9 ml of diluent at pH 7.0, and then three successive dilutions were made. Samples from each dilution were plated in duplicate and agar was added. For each treatment, one ml of the previous mixture was added to 9 ml of neutralizing solution and then mixed. One ml of the neutralized solution was then placed into petri dishes in duplicate. Approximately 15 ml of sterile tryptic soy agar was added to each Petri dish and when solidified, each plate was incubated at 30° C. for 48 hours. Colony forming units on plates were counted after 48 hours incubation. This procedure was repeated for all samples to be tested. Passing yeasticidal efficacy is at least a 4-log reduction of starting inoculum cell counts.

The plates with microbial growth populations between 30 and 300 were counted and results were expressed as logarithmic reductions according to EN 1656 and EN 1657 test methods. The tables below provides the results of the EN 1656 and EN 1657 tests as well as the active concentration of each chemical used in the formulation.

The data shown in Tables 2 and 3 generally indicate that preferred compositions having lactic acid concentrations of greater than 0.5%, salicylic acid concentrations of 0.1% or greater, and SLS concentrations of 0.5% or greater exhibit acceptable yeasticidal and antibacterial characteristics.

TABLE 2

| Ingredient | 1 | 2 | 3 | 4 | 5 | 6 | 7 | 8 | 9 | 10 | 11 | 12 |
|---|---|---|---|---|---|---|---|---|---|---|---|---|
| Deionized Water | 65.30 | 64.57 | 71.97 | 72.02 | 69.62 | 72.12 | 72.51 | 72.76 | 73.26 | 72.02 | 74.26 | 75.37 |
| Sorbitol, 70% | 11.42 | 11.42 | 11.42 | 11.42 | 11.42 | 11.42 | 11.42 | 11.42 | 11.42 | 11.42 | 11.42 | 11.42 |
| Pullulan | 0.30 | 0.30 | 0.30 | 0.30 | 0.30 | 0.30 | 0.30 | 0.30 | 0.30 | 0.30 | 0.30 | 0.30 |
| Xanthan gum | 0.40 | 0.40 | 0.40 | 0.40 | 0.40 | 0.40 | 0.40 | 0.40 | 0.40 | 0.40 | 0.40 | 0.40 |
| L(+)-Lactic acid, HS, | 3.92 | 3.92 | 3.92 | 3.92 | 3.92 | 3.92 | 3.33 | 3.33 | 3.33 | 3.92 | 2.22 | 1.11 |
| 90% | | | | | | | | | | | | |
| Poloxamer 338 | 0.20 | 0.20 | 0.20 | 0.20 | 0.20 | 0.20 | 0.20 | 0.20 | 0.20 | 0.20 | 0.20 | 0.20 |
| (Pluronic F108) | | | | | | | | | | | | |
| Allantoin | 0.10 | 0.10 | 0.10 | 0.10 | 0.10 | 0.10 | 0.10 | 0.10 | 0.10 | 0.10 | 0.10 | 0.10 |
| Magnesium oxide | 0 | 0.10 | 0 | 0.05 | 0.05 | 0.05 | 0.05 | 0.05 | 0.05 | 0.05 | 0.050 | 0.050 |
| Lanolin 50% | 4.00 | 4.00 | 4.00 | 4.00 | 4.00 | 4.00 | 4.00 | 4.00 | 4.00 | 4.00 | 4.00 | 4.00 |
| E133 Granular | 0.0036 | 0.0036 | 0.0036 | 0.0036 | 0.0036 | 0.0036 | 0.0036 | 0.0036 | 0.0036 | 0.0036 | 0.0036 | 0.0036 |
| (colorant) | | | | | | | | | | | | |
| E102 Granular | 0.015 | 0.015 | 0.015 | 0.015 | 0.015 | 0.015 | 0.015 | 0.015 | 0.015 | 0.015 | 0.015 | 0.015 |
| (colorant) | | | | | | | | | | | | |
| Sodium | 0.15 | 0.15 | 0.15 | 0.15 | 0.15 | 0.15 | 0.15 | 0 | 0 | 0.15 | 0 | 0 |
| dioctylsulfosuccinate, | | | | | | | | | | | | |
| 75% (Aerosol OT) | | | | | | | | | | | | |
| Alpha olefin | 0 | 7.00 | 0 | 0 | 2.50 | 0 | 0 | 0 | 0 | 0 | 0 | 0 |
| sulfonate, 40% | | | | | | | | | | | | |
| Sodium lauryl sulfate, | 10 | 3.33 | 3.33 | 3.33 | 3.33 | 3.33 | 3.33 | 3.33 | 3.33 | 3.33 | 3.33 | 3.33 |
| 30% | | | | | | | | | | | | |
| Propylene glycol | 3.00 | 3.00 | 3.00 | 3.00 | 3.00 | 3.00 | 3.00 | 3.00 | 3.00 | 3.00 | 3.00 | 3.00 |
| Salicylic acid | 0 | 0.40 | 0.40 | 0.40 | 0.20 | 0.20 | 0.20 | 0.20 | 0.20 | 0.20 | 0.20 | 0.20 |
| TWEEN 80 | 0.30 | 0.30 | 0.30 | 0.30 | 0.30 | 0.30 | 0.30 | 0.30 | 0.00 | 0.30 | 0.30 | 0.30 |
| Sodium hydroxide, | 0.9 | 0.8 | 0.5 | 0.4 | 0.5 | 0.5 | 0.7 | 0.6 | 0.4 | 0.6 | 0.20 | 0.20 |
| 29% | | | | | | | | | | | | |
| Log Reduction, | 5.9 | 5.4 | 6.5 | 6.5 | 5.4 | 5.7 | 4.5 | 5.7 | 5.7 | 5.7 | 5.5 | 5.1 |
| C. albicans, EN 1657, | | | | | | | | | | | | |
| 30° C., milk, 5 min | | | | | | | | | | | | |
| Log Reduction, | — | — | — | — | 6.3 | 6.3 | 6.3 | 6.3 | 6.3 | 6.3 | 6.5 | 6.5 |
| S. aureus, EN 1656, | | | | | | | | | | | | |
| 30° C., milk, 5 min | | | | | | | | | | | | |
| Log Reduction, | — | — | — | — | 6.3 | 6.3 | 6.3 | 6.3 | 6.3 | 6.3 | 6.7 | 6.1 |
| E. coli, EN 1656, | | | | | | | | | | | | |
| 30° C., milk, 5 min | | | | | | | | | | | | |
| Log Reduction | — | — | — | — | 6.5 | 6.5 | 6.5 | 6.5 | 6.5 | 6.5 | 6.2 | 6.2 |
| S. uberis, EN 1656, | | | | | | | | | | | | |
| 30° C., milk, 5 min | | | | | | | | | | | | |

Formula 1, which did not comprise MgO or salicylic acid, while exhibiting yeasticidal characteristics, was discovered to not be physically stable at 4° C.

TABLE 3

| Ingredient | 11 | 12 | 13 | 14 | 15 | 16 | 17 | 18 | 19 | 20 | 21 |
|---|---|---|---|---|---|---|---|---|---|---|---|
| Deionized Water | 75.93 | 72.71 | 72.86 | 73.01 | 73.16 | 73.31 | 73.34 | 76.05 | 73.13 | 64.48 | 75.66 |
| Sorbitol, 70% | 11.42 | 11.42 | 11.42 | 11.42 | 11.42 | 11.42 | 11.42 | 11.42 | 11.42 | 11.42 | 11.42 |
| Pullulan | 0.30 | 0.30 | 0.30 | 0.30 | 0.30 | 0.30 | 0.30 | 0.30 | 0.30 | 0.30 | 0.30 |
| Xanthan gum | 0.40 | 0.40 | 0.40 | 0.40 | 0.40 | 0.40 | 0.40 | 0.40 | 0.40 | 0.40 | 0.40 |
| L(+)-Lactic acid, HS, | 0.56 | 3.33 | 3.33 | 3.33 | 3.33 | 3.33 | 3.33 | 3.33 | 5.56 | 11.11 | 3.33 |
| 90% | | | | | | | | | | | |
| Poloxamer 338 | 0.20 | 0.20 | 0.20 | 0.20 | 0.20 | 0.20 | 0.20 | 0.20 | 0.20 | 0.20 | 0.20 |
| (Pluronic F108) | | | | | | | | | | | |
| Allantoin | 0.10 | 0.10 | 0.10 | 0.10 | 0.10 | 0.10 | 0.10 | 0.10 | 0.10 | 0.10 | 0.10 |
| Magnesium oxide | 0.050 | 0.05 | 0.05 | 0.05 | 0.05 | 0.05 | 0.05 | 0.05 | 0.05 | 0.05 | 0.05 |
| Lanolin 50% | 4.00 | 4.00 | 4.00 | 4.00 | 4.00 | 4.00 | 4.00 | 4.00 | 4.00 | 4.00 | 4.00 |
| E133 Granular | 0.0036 | 0.0036 | 0.0036 | 0.0036 | 0.0036 | 0.0036 | 0.0036 | 0.0036 | 0.0036 | 0.0036 | 0.0036 |
| (colorant) | | | | | | | | | | | |
| E102 Granular | 0.015 | 0.015 | 0.015 | 0.015 | 0.015 | 0.015 | 0.015 | 0.015 | 0.015 | 0.015 | 0.015 |
| (colorant) | | | | | | | | | | | |
| Sodium | 0 | 0.15 | 0 | 0.15 | 0 | 0 | 0 | 0 | 0 | 0 | 0 |
| dioctylsulfosuccinate, | | | | | | | | | | | |
| 75% (Aerosol OT) | | | | | | | | | | | |
| Alpha olefin | 0 | 0 | 0 | 0 | 0 | 0 | 0 | 0 | 0 | 0 | 0 |
| sulfonate, 40% | | | | | | | | | | | |
| Sodium lauryl sulfate, | 3.33 | 3.33 | 3.33 | 3.33 | 3.33 | 3.33 | 3.33 | 3.33 | 3.33 | 3.33 | 0.83 |
| 30% | | | | | | | | | | | |
| Propylene glycol | 3.00 | 3.00 | 3.00 | 3.00 | 3.00 | 3.00 | 3.00 | 0.00 | 0.00 | 3.00 | 3.00 |
| Salicylic acid | 0.20 | 0.20 | 0.20 | 0.20 | 0.20 | 0.10 | 0.05 | 0.20 | 0.50 | 0.20 | 0.20 |
| TWEEN 80 | 0.30 | 0.30 | 0.30 | 0.30 | 0.30 | 0.30 | 0.30 | 0.30 | 0.30 | 0.30 | 0.30 |
| Sodium hydroxide, | 0.20 | 0.5 | 0.5 | 0.2 | 0.2 | 0.15 | 0.17 | 0.31 | 0.7 | 1.1 | 0.2 |
| 29% | | | | | | | | | | | |
| Log Reduction, | <3.0 | 5.7 | 5.7 | 5.5 | 5.5 | 5.3 | 4.6 | 5.6 | 5.6 | 4.2 | 5.3 |
| C. albicans, EN 1657, | | | | | | | | | | | |
| 30° C., milk, 5 min | | | | | | | | | | | |
| Log Reduction, | 6.5 | 6.3 | 6.3 | 6.3 | 6.3 | 6.6 | 6.6 | 6.3 | 6.3 | 6.6 | 6.6 |
| S. aureus, EN 1656, | | | | | | | | | | | |
| 30° C., milk, 5 min | | | | | | | | | | | |
| Log Reduction, | 5.1 | 6.3 | 6.3 | 6.3 | 6.3 | 6.4 | 6.4 | 6.5 | 5.3 | 6.4 | 6.4 |
| E. coli, EN 1656, | | | | | | | | | | | |
| 30° C., milk, 5 min | | | | | | | | | | | |
| Log Reduction | 6.2 | 6.5 | 6.5 | 6.5 | 6.5 | 6.2 | 6.2 | 6.1 | 6.1 | 6.2 | 6.2 |
| S. uberis, EN 1656, | | | | | | | | | | | |
| 30° C., milk, 5 min | | | | | | | | | | | |

TABLE 4

| Ingredient | 22 | 23 | 24 | 25 | 26 |
|---|---|---|---|---|---|
| Deionized Water | 74.00 | 74.75 | 69.74 | 60.49 | 66.84 |
| Sorbitol, 70% | 11.42 | 11.42 | 11.42 | 11.42 | 11.42 |
| Pullulan | 0.30 | 0.30 | 0.30 | 0.30 | 0.30 |
| Xanthan gum | 0.40 | 0.40 | 0.40 | 0.40 | 0.40 |
| L(+)-Lactic acid, HS, | 3.33 | 3.33 | 3.33 | 3.33 | 3.33 |
| 90% | | | | | |
| Poloxamer 338 | 0.20 | 0.20 | 0.20 | 0.20 | 0.20 |
| (Pluronic F108) | | | | | |
| Allantoin | 0.10 | 0.10 | 0.10 | 0.10 | 0.10 |
| Magnesium oxide | 0.050 | 0.050 | 0.050 | 0.050 | 0.050 |
| Lanolin 50% | 4.00 | 4.00 | 4.00 | 4.00 | 4.00 |
| E133 Granular | 0.0036 | 0.0036 | 0.0036 | 0.0036 | 0.0036 |
| (colorant) | | | | | |
| E102 Granular | 0.015 | 0.015 | 0.015 | 0.015 | 0.015 |
| (colorant) | | | | | |
| Sodium | 0 | 0 | 0 | 0 | 0 |
| dioctylsulfosuccinate, | | | | | |
| 75% (Aerosol OT) | | | | | |
| Alpha olefin | 0 | 0 | 0 | 0 | 0 |
| sulfonate, 40% | | | | | |
| Sodium lauryl sulfate, | 3.33 | 3.33 | 6.66 | 9.99 | 6.66 |
| 30% | | | | | |
| Propylene glycol | 2.00 | 1.00 | 3.00 | 9.00 | 6.00 |
| Salicylic acid | 0.20 | 0.20 | 0.20 | 0.20 | 0.20 |
| TWEEN 80 | 0.30 | 0.30 | 0.30 | 0.30 | 0.30 |
| Sodium hydroxide, | 0.35 | 0.60 | 0.28 | 0.20 | 0.18 |
| 29% | | | | | |
| Ratio of parts | 1:2 | 1:1 | 2:3 | 3:9 | 2:6 |
| SLS to parts | | | | | |
| propylene glycol | | | | | |
| Log Reduction, | 5.5 | 4.3 | 5.5 | 5.2 | 5.5 |
| C. albicans, EN 1657, | | | | | |
| 30° C., milk, 5 min | | | | | |
| Log Reduction, | 6.5 | 4.9 | 6.5 | 6.5 | 6.5 |
| S. aureus, EN 1656, | | | | | |
| 30° C., milk, 5 min | | | | | |
| Log Reduction, | 6.7 | 6.7 | 6.7 | 6.7 | 6.7 |
| E. coli, EN 1656, | | | | | |
| 30° C., milk, 5 min | | | | | |
| Log Reduction | 6.2 | 6.2 | 6.2 | 6.2 | 6.2 |
| S. uberis, EN 1656, | | | | | |
| 30° C., milk, 5 min | | | | | |

Formulations 24-26 were not storage stable for 3 weeks at 4 C, despite being antimicrobially effective. This indicates that for the formulations tested, it becomes difficult to keep the SLS dissolved when it is present in the formulation in concentrations greater than 2% by weight. However, the data also shows that a minimum ratio of SLS to propylene glycol is required to achieve acceptable yeasticidal and bactericidal performance. In the embodiments tested, a ratio of SLS to propylene glycol of less than 1:1 (i.e., a greater amount of propylene glycol relative to SLS) is required in order to achieve acceptable antimicrobial performance. Without wishing to be bound by any theory, it is believed that the to combination of SLS and propylene glycol reduces barriers to cell permeation, possibly allowing lactic acid easier access to the interior of the bacteria and yeast cells.

TABLE 5

| Ingredient | 27 | 28 | 29 | 30 |
|---|---|---|---|---|
| Deionized water | 72.91 | 65.57 | 75.13 | 72.52 |
| Sorbitol, 70% | 11.42 | 11.42 | 11.42 | 11.42 |
| Pullulan | 0.30 | 0.30 | 0.30 | 0.30 |
| Xanthan gum | 0.40 | 0.40 | 0.40 | 0.40 |
| L(+)-Lactic Acid, HS, | 3.33 | 3.33 | 3.33 | 3.33 |
| 90% | | | | |
| Poloxamer 338 | 0.20 | 0.20 | 0.20 | 0.20 |
| (Pluronic F108) | | | | |
| Allantoin | 0.10 | 0.10 | 0.10 | 0.10 |
| Magnesium oxide | 0.05 | 0.05 | 0.05 | 0.05 |
| Lanolin 50% | 4.00 | 4.00 | 4.00 | 4.00 |
| E133 Granular | 0.0036 | 0.0036 | 0.0036 | 0.0036 |
| (colorant) | | | | |
| E102 Granular | 0.015 | 0.015 | 0.015 | 0.015 |
| (colorant) | | | | |
| Sodium | 0 | 0 | 0 | 0 |
| dioctylsulfosuccinate, | | | | |
| 75% (Aerosol OT) | | | | |
| Alpha olefin | 0 | 0 | 0 | 0 |
| sulfonate, 40% | | | | |
| Sodium lauryl sulfate, | 0 | 0 | 0 | 0 |
| 30% | | | | |
| Propylene glycol | 3.00 | 3.00 | 3.00 | 3.00 |
| Salicylic acid | 0.20 | 0.20 | 0.20 | 0.20 |
| TWEEN 80 (Eur. Ph.) | 0.30 | 0.30 | 0.30 | 0.30 |
| Sodium hydroxide, | 0.20 | 0.40 | 0.50 | 1.00 |
| 29% | | | | |
| Sodium lauryl ether sulfate, | 3.57 | 10.71 | 0 | 0 |
| 28% | | | | |
| Multitrope 1214, 95% | 0 | 0 | 1.05 | 3.16 |
| (Alkoxylated phosphate | | | | |
| ester) | | | | |
| Log Reduction, | 5.4 | 5.4 | 5.4 | 5.4 |
| C. albicans, EN 1657, | | | | |
| 30° C., milk, 5 min | | | | |
| Log Reduction, | 6.5 | 6.5 | 6.5 | 6.5 |
| S. aureus, EN 1656, | | | | |
| 30° C., milk, 5 min | | | | |
| Log Reduction, | 6.1 | 6.1 | 6.1 | 6.1 |
| E. coli, EN 1656, | | | | |
| 30° C., milk, 5 min | | | | |
| Log Reduction | 6.3 | 6.3 | 6.3 | 6.3 |
| S. uberis, EN 1656, | | | | |
| 30° C., milk, 5 min | | | | |

1. An antimicrobial composition comprising:
from 0.75% to 10% by weight of lactic acid;
from 0.01% to 5% by weight of salicylic acid;
from 0.1% to 15% by weight of at least one anionic surfactant selected from the group consisting of polyoxyethylene alkyl ether phosphates, sodium lauryl sulfate, and sodium lauryl ether sulfate.

2. The composition according to claim 1, further comprising from 0.01% to 1% by weight of magnesium oxide.

3. The composition according to claim 1, further comprising from 0.01% to 5% of one or more nonionic surfactants, wherein at least one of the nonionic surfactants comprises a polyoxyethylene, polyoxypropylene block polymer or polyoxyethylene sorbitan monooleate.

4. The composition according to claim 3, further comprising from 0.5% to 10% by weight of propylene glycol.

5. The composition according to claim 4, wherein the ratio of nonionic surfactant to propylene glycol is less than 1:1.

6. The composition according to claim 1, wherein the anionic surfactant is sodium lauryl sulfate.

7. The composition according to claim 6, wherein the amount of sodium lauryl sulfate is 0.1-5% by weight.

8. The composition according to claim 1, further comprising from 5% to 20% by weight of sorbitol.

9. The composition according to claim 1, wherein the amount of lactic acid is 1-7.5% by weight.

10. The composition according to claim 1, wherein the amount of salicylic acid is 0.05-2.5% by weight.

11. The composition according to claim 1, wherein the total amount of anionic surfactant is 0.2-10% by weight.

12. A method of treating the teats of a bovine comprising applying to the teats of the bovine a teat dip composition comprising the antimicrobial composition of claim 1, the teat dip composition being effective in reducing the levels of bacteria and yeast present on the teats.
